(19) United States
(12) Patent Application Publication
(10) Pub. No.: US 2025/0001075 A1
(43) Pub. Date: Jan. 2, 2025

(54) INFUSION PUMP HAVING A BACKUP POWER SYSTEM

(71) Applicant: B. BRAUN MEDICAL INC., Bethlehem, PA (US)

(72) Inventors: Hans Schmolk, Perkasie, PA (US); Michael Robert Pavlovic, Stroudsburg, PA (US)

(73) Assignee: B. BRAUN MEDICAL INC., Bethlehem, PA (US)

(21) Appl. No.: 18/216,486

(22) Filed: Jun. 29, 2023

(51) Int. Cl. A61M 5/172

(52) U.S. Cl. CPC A61M 5/1723; A61M 2205/52; A61M 2205/82

(57) ABSTRACT

An infusion pump includes a controller that controls the pump's operating states including an initiation state and a current operating state for an infusion. The infusion pump has a clock that tracks the time of the current operating state of the infusion and a memory in communication with the controller and the clock that stores pump settings. The infusion pump includes a primary power source that provides power to the controller, clock, memory, and other pump components required for pumping during an infusion. The pump also includes a backup power source that provides power to the controller, clock, and memory during interruptions in power from the primary power source.

### FIELD OF THE INVENTION

The present disclosure is related to infusion pumps and, more particularly, to infusion pumps having backup power systems.

### BACKGROUND

Infusion pumps deliver controlled doses of fluids such as medications, analgesics, and nutrition to patients. Infusion pumps are particularly well suited to delivering controlled doses of fluids over long periods of time, e.g., several hours or days. While many infusion pumps are designed for bedside use, there are ambulatory versions available. Ambulatory infusion pumps allow a patient to move around while the infusion pump is in use.

Syringe pumps and peristaltic pumps are two conventional types of infusion pumps. A syringe pump depresses a cylinder within a syringe to deliver fluid from the syringe to a patient. A peristaltic pump acts on a tube to control the rate of fluid flow through the tube from a bottle or bag of fluid to a patient. Precise delivery of fluids are desirable to optimize treatment of a patient as there are many fluids where small variations can be critical.

Infusion pumps are typically powered by an electrical power source. However, an electrical failure may interrupt an infusion. An improperly reinitiated infusion upon restoration of power may result in patient harm.

### SUMMARY

Examples described herein are directed to infusion methods and infusion pumps for delivering fluids to a patient. In sample configurations, an infusion pump is described that includes a pump configured to deliver an infusion, a clock configured to track time, a memory, and a controller. The controller is in operative communication with the pump, the clock, and the memory and is configured to control the infusion pump's operation, retrieve time from the clock, and store pump settings (including a duration of the infusion) in the memory. The infusion pump additionally includes a primary power source configured to provide power to the pump, the clock, the memory, and the controller during the infusion and a backup power source configured to provide power to the clock and the memory during an interruption in power from the primary power source to maintain the pump settings.

A method of operating an infusion pump is also described. The method includes providing an infusion using the infusion pump, tracking time with a clock, storing pump settings for the infusion in a memory, the pump settings including a duration of the infusion, detecting interruption in power from a primary power source for the infusion pump, suspending the infusion upon detecting the interruption in power from the primary power source, and powering the clock and the memory with a backup power source while the infusion is suspended to maintain the pump settings for the infusion.

A non-transitory controller-readable storage medium storing controller-executable instructions that controls the operation of an infusion pump is also described. The medium stores instructions that when executed by the infusion pump's controller causes the infusion pump described herein to perform operations that include providing an infusion using the infusion pump, tracking time with a clock, storing pump settings for the infusion in a memory, the pump settings including a duration of the infusion, detecting interruption in power from a primary power source for the infusion pump, suspending the infusion upon detecting the interruption in power from the primary power source, and powering the clock and the memory with a backup power source while the infusion is suspended to maintain the pump settings for the infusion.

### BRIEF DESCRIPTION OF THE DRAWINGS

The drawing figures depict multiple views of one or more implementations, by way of example only, not by way of limitations. In the figures, like reference numerals refer to the same or similar elements. The same numeral is used to represent the same or similar element across the multiple views. If multiple elements of the same or similar type are present, a letter may be used to distinguish between the multiple elements. When the multiple elements are referred to collectively or a non-specific one of the multiple elements is being referenced, the letter designation may be dropped.

FIG. 1 is a perspective view of an example ambulatory peristaltic infusion pump.

FIG. 2 is a perspective view of an example cassette with a free flow prevention clamp for use with the ambulatory peristaltic infusion pump of FIG. 1.

FIG. 3 is a partial perspective view of the ambulatory peristaltic infusion pump of FIG. 1 illustrating cams that engage the free flow prevention clamp when the cassette is coupled to the ambulatory peristaltic infusion pump.

FIGS. 4 and 5 are cutaway views of the ambulatory peristaltic infusion pump of FIG. 1 illustrating pump fingers and cams for moving the pump fingers.

FIG. 6A is a schematic block diagram of the circuitry of the infusion pump providing an overview of the supervisor controller according to one example of the present disclosure.

FIG. 6B is a schematic block diagram of the circuitry of the primary power source of the infusion pump of FIG. 6A according to one example of the present disclosure.

FIG. 7 is a flow chart illustrating the operation of the primary and backup power sources according to an example of the present disclosure.

FIG. 8 is a functional block diagram illustrating a general-purpose computer hardware platform configured to implement the functional examples described with respect to FIGS. 1-7.

FIG. 9 is another functional block diagram illustrating a general-purpose computer hardware platform configured to implement the functional examples described with respect to FIGS. 1-7.

### DETAILED DESCRIPTION

In the following detailed description, numerous specific details are set forth by way of examples to provide a thorough understanding of the relevant teachings. However, it should be apparent to those skilled in the art that the present teachings may be practiced without such details. In other instances, well known methods, procedures, components, and circuitry have been described at a relatively high-level, without detail, in order to avoid unnecessarily obscuring aspects of the present teachings. Moreover, while described with respect to an ambulatory peristaltic infusion pump for pain management, homecare, outpatient infusions, and the like, it will be appreciated by those skilled in the art that the backup power source described herein may be used with a variety of other pump types.

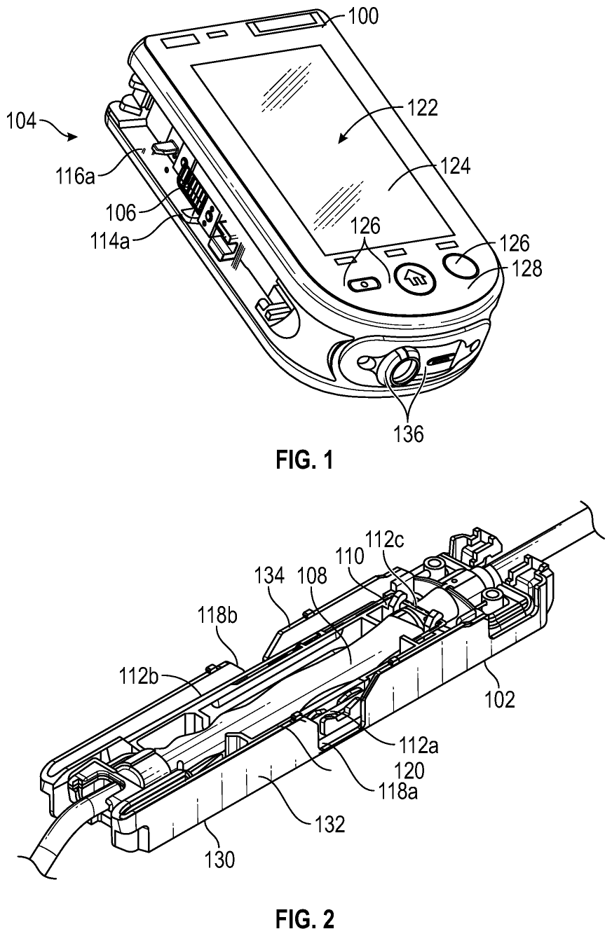

FIG. 1 depicts an example ambulatory peristaltic infusion pump 100, while FIG. 2 depicts an example cassette 102 for use with the infusion pump 100. The infusion pump 100 includes a receptacle 104 configured to receive the cassette 102. A peristaltic pump 106 within the receptacle 104 acts upon a tube 108 extending through a channel within the cassette 102 to pump fluid from a fluid container (e.g., a bag or a bottle; not shown) into a patient. An example free flow prevention clamp 110 is positioned within the cassette 102 to allow fluid flow through the tube 108 when the cassette 102 is coupled to the infusion pump 100 within the receptacle 104 (during which time the peristaltic pump 106 controls fluid flow through the tube 108) and to selectively cut off fluid flow through the tube 108 when the cassette 102 is not coupled to the infusion pump 100 in order to prevent unintentional fluid flow through the tube 108 (e.g., free flow).

The infusion pump 100 includes a user interface 122 for interacting with the infusion pump 100. The illustrated user interface 122 includes a display 124 (which may be a touchscreen) and buttons 126. A user controls operation of the infusion pump 100 via the user interface 122. The infusion pump 100 additionally includes a housing 128 for containing and supporting the components of the infusion pump 100 such as the peristaltic pump 106, electronics, power supplies, and the like.

The free flow prevention clamp 110 includes a first elongate section 112a, a second elongate section 112b, and a clamping section 112c. The housing 130 of the cassette 102 supports the free flow prevention clamp 110. The clamping section 112c is positioned within the cassette geometry such that, when the cassette 102 is received within the receptacle 104 of the infusion pump 100, the clamping section 112c extends across the channel receiving the tube 108. The housing 130 of the cassette 102 may be rigid plastic or other material capable of supporting the tube 108 and free flow prevention clamp 110.

The infusion pump 100 also includes a pair of arc cams 114a and 114b. First arc cam 114a is shown on one side of the receptacle illustrated in FIG. 1, but the second arc cam 114b is hidden from view. The pair of arc cams 114a and 114b engage the elongate sections 112a, 112b of the free flow prevention clamp 110 in order to lift the clamping section 112c. Additionally, the infusion pump 100 includes a pair of wedge cams 116a and 116b. A first wedge cam 116a is shown on one side of the receptacle 104 illustrated in FIG. 1, but the second wedge cam 116b is hidden from view. The pair of wedge cams 116a and 116b transition the free flow prevention clamp 110 from an open, manufactured/shipped state to an operational state, which is described in further detail below.

The cassette 102 also includes a first cutout 118a in a sidewall 132 of the cassette 102 and a second cutout 118b in an opposite sidewall 134 of the cassette 102. Additionally, the cassette 102 includes a touch pad 120 positioned on the first elongate section 112a adjacent a mid-point of the first elongate section 112a and the first cutout 118a. The touch pad 120 and cutout 118a together facilitate engagement of the first elongate section 112a by a finger of an operator in order to manually lift the clamping section 112c to allow fluid flow through the tube 108 (e.g., for priming the cassette 102) when the cassette 102 is not received within the receptacle 104 of the infusion pump 100. The touch pad 120 may be a press fit piece of rigid plastic. Although the touch pad 120 is illustrated as only on the first elongate section 112a, the touch pad 120 also may be provided on the second elongate section 112b.

The ambulatory infusion pump 100 further includes connector ports 136 that provide electronic access for control and for powering the ambulatory infusion pump 100 when used in one or more of the configurations described below.

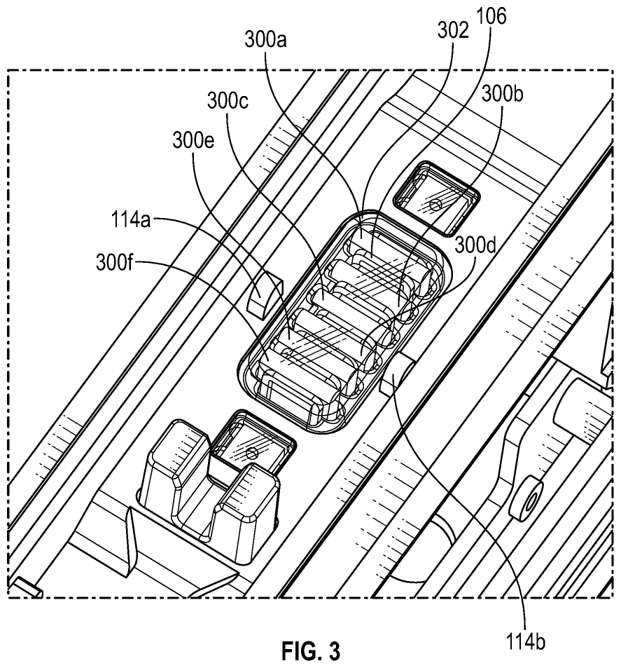

FIG. 3 depicts the arc cams 114a and 114b and the peristaltic pump 106 of the infusion pump 100. The peristaltic pump 106 includes multiple pump fingers 300 (six pump fingers 300a-f illustrated in FIG. 3). A flexible barrier 302 separates the pump fingers 300 (and other pump components of a pumping mechanism) from the receptacle 104 receiving the cassette 102 with the tube 108. The flexible barrier 302 provides a barrier between the fluid delivery apparatus/cassette and the pumping mechanism to prevent fluid from damaging components of the pumping mechanism.

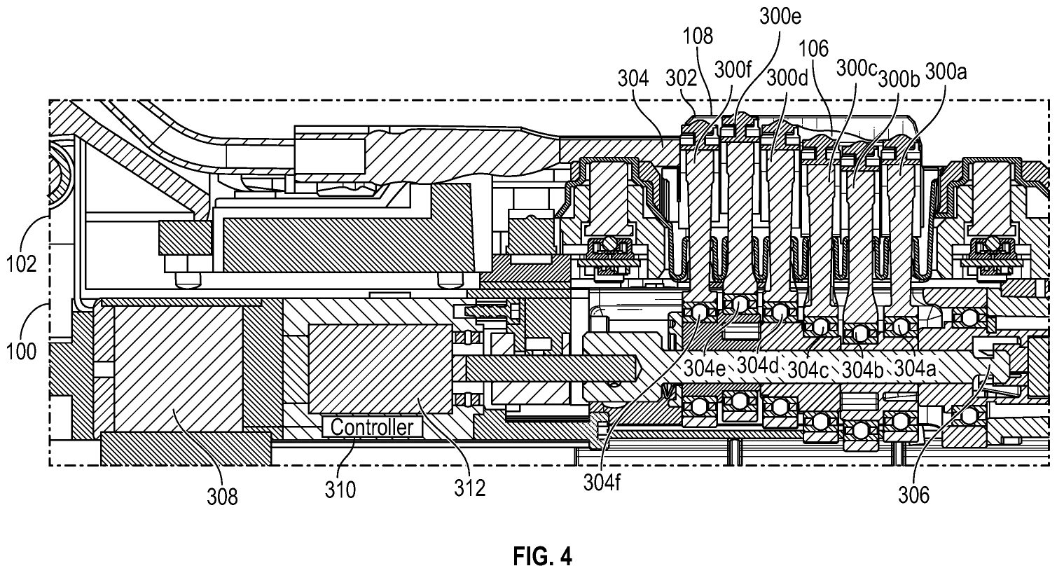

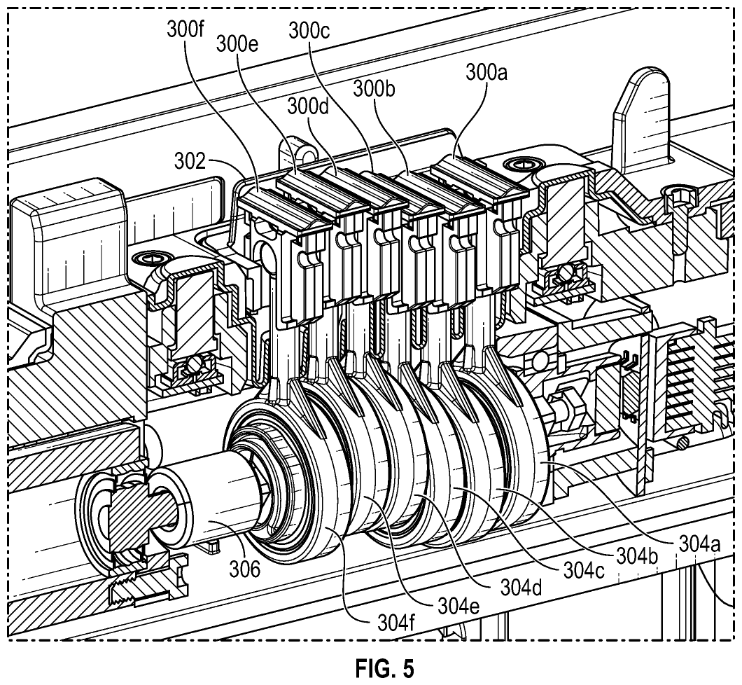

FIGS. 4 and 5 are cutaway views of the infusion pump 100 with the cassette 102 inserted into the receptacle 104 of the ambulatory infusion pump 100. Multiple cams 304 (six cams 304a-f), supported by a camshaft 306, act on respective pump fingers 300a-300f. The cams 304a-f respectively raise and lower the pump fingers 300a-f, which engage the tube 108 of the cassette 102 in order to force fluid though the tube 108. A pump motor 308 under control of a control system 310 turns the camshaft 306 by way of a gearbox 312. As the camshaft 306 turns, the cams 304a-300f, which are offset from each other in an axial direction, raise and lower respective pump fingers 300a-300f For example, cam 304a raises and lowers pump finger 300a; cam 304b raises and lowers pump finger 300b, and the like. The control system 310 may be a standalone or embedded processing system configured to carry out instructions in order to control operations of the infusion pump 100.

The control system 310 may include a user interface main controller core/system controller core such as a dual core 32 bit processor from NXP of Eindhoven, Netherlands (e.g., model #MCIMX7S5EVM08SC), a pump controller core from NXP (e.g., model #MKV31F512VLH12), a supervisor controller core from NXP (e.g., model #MKL17Z128VMP4), a pump motor driver from ST Microelectronics of Geneva, Switzerland (e.g., model #STSPIN250), and a magnetic encoder from Austriamicrosystems of Premstaetten, Austria (e.g., model number AS5601-ASOM). The microcontroller receives pump camshaft revolutions per minute (RPM) corresponding to the infusion rate from a system controller core of the main processor. The microcontroller develops a pulse width modulation (PWM) motor drive parameter relating to the desired camshaft RPM. The PWM output of the microcontroller becomes the motor drive input to the pump motor driver, which contains motor drive transistors and protection circuitry. The rotation of the camshaft 306 of the pumping mechanism is measured by the magnetic encoder. At specified time intervals, the output of the encoder is read by the microcontroller, which uses the encoder value to compute the speed of the camshaft 306 and the position of the pump rotation. These values are then used to modify the PWM output to maintain the correct camshaft RPM.

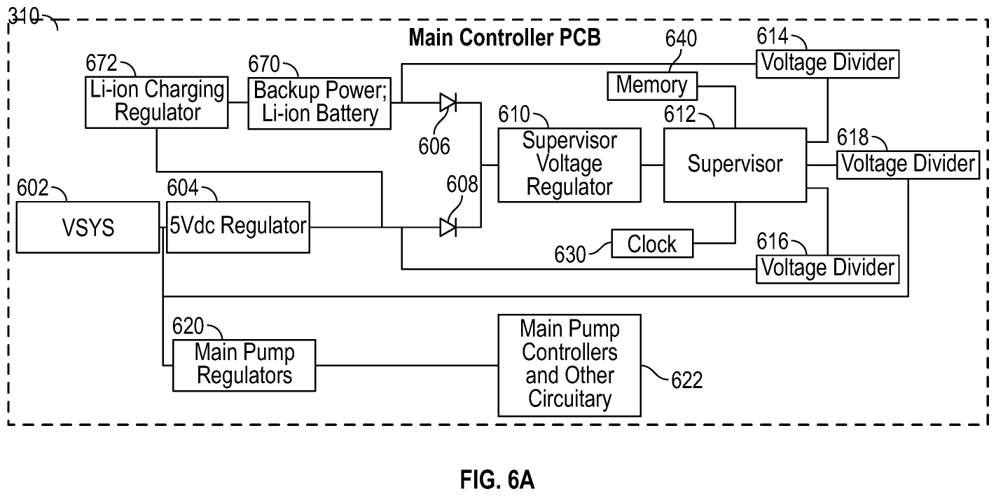

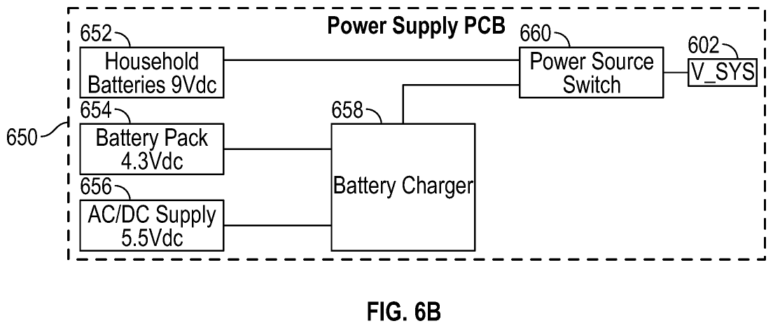

FIG. 6A is a schematic block diagram illustrating components of the control system 310 of the infusion pump 100. During normal operation (e.g., when primary power is available), the illustrated control system 310 is powered by a system voltage 602 received from primary power source 650 (described below with reference to FIG. 6B) and, during a power interruption (e.g., when primary is not available) the control system is powered by a backup power source 670. The control system 310 detects interruptions in power from primary power source 650 and selectively powers components (e.g., on a main controller printed circuit board (PCB)) during power interruptions to maintain time and pump parameters.

A voltage regulator 604 regulates the system voltage 602 to provide a regulated voltage (e.g., 5 volts) for powering components of the control system 310. The regulated voltage charges the backup power supply 670 via a charging regulator 672. In one example, the backup power source 670 is a rechargeable Lithium Ion (Li-Ion) battery and the voltage regulator 604 is a conventional Li-Ion charging regulator.

The primary power source 650 provides power to the circuitry of the pump during normal/uninterrupted power operation. The power for primary power source 650 is drawn from a main power source (household batteries, battery pack, DC power from AC/DC converter, etc.) that is stepped down to a working voltage level by voltage regulator 604 and delivered to a junction that also receives a voltage input developed from the backup power source 670 (via respective diodes 606 and 608 that prevent backflow of current). The junction, e.g., an OR'ed junction (not shown), provides an input voltage to a supervisor controller voltage regulator 610 that provides a regulated voltage level (e.g., 3.5 volts) to a supervisor microcontroller 612.

Backup power source 670 includes one or more energy storage devices such as capacitors, backup batteries, or the like to provide temporary power to maintain the memory and the clock in the event of interruption of power supplied by the primary power source 650. The backup power source 670 may comprise a rechargeable or non-rechargeable battery as described above for primary power source 650 or may be powered by alkaline, nickel cadmium, lithium, or lithium-ion batteries, or alternatively is a photovoltaic power source, thermoelectric power source and the like. Different types of batteries exhibit different voltage characteristics over time. In an example, voltage regulator 604 is connected operatively between the backup power source 670 and the supervisor controller 612. In one aspect, the voltage regulator 604 comprises a capacitor for stabilizing the output voltage of the voltage regulator.

Examples of a primary power interruption/failure may include but are not limited to the interruption in the conductance of electricity through the wired connection, removal of the primary power supply, failing of a cell within the primary power supply, and the failing of a conductor that electrically-couples the primary power supply to the controller. Accordingly, in the event of such a failure, primary power supply may no longer provide primary electrical energy to the controller. Suitable backup power sources 670 include a battery pack (conventional or rechargeable). Example rechargeable or non-rechargeable batteries include common AA or AAA alkaline cell or a special-purpose power pack. In one example the backup power source is a rechargeable battery such as a lithium ion rechargeable battery.

In one example, the junction between the primary power source 650 and the backup power source 670 is a wired OR junction. In accordance with this example, the voltage of the backup power source 670 is applied to the anode of the diode 606 and the voltage of the primary power source 650 (after regulation by regulator 604) is applied to the anode of the diode 608. The cathodes of the diode 606 and the diode 608 are “wire OR'ed” together to provide input to the voltage regulator 610 for delivery to supervisor controller 612. When primary power is available/active, the voltage level developed at the junction is higher than the voltage level output of the backup power source 670. This reverse biases the diode 606 (non-active) while the diode 608 is forward biased (active) to provide the power from the primary power source 670 to the voltage regulator 610 for the supervisor controller 612. When primary power 650 is interrupted/non-active, the voltage level provided by the primary power 602 through the regulator 604 is lower than the voltage output of the backup power source 670. This causes the diode 608 to become reverse biased (non-active) and the diode 606 to become forward biased (active), which now provides the input voltage from the backup power source 670 to the voltage regulator 610 for the supervisor controller 612 to seamlessly maintain power.

The supervisor controller 612 monitors the primary system voltage level (system voltage 602 via voltage divider 618 and as stepped down by regulator 604 via voltage divider 616) and the backup battery voltage level (via voltage divider 614). It is notable that monitoring both system voltage 602 via voltage divider 618 and as stepped down by regulator 604 via voltage divider 616 provides redundancy for determining whether there is an interruption in the system voltage 602 (as opposed to a component error or failure condition such as a defective voltage regulator). Accordingly, the supervisor controller 612 may determine that the primary power source 650 is active if one or both of the voltage dividers 616 and 618 are presenting an expected voltage to the supervisor controller 612 and that the primary power source 650 is interrupted only when both voltage dividers 616 and 618 are presenting a voltage level to the supervisor controller 612 that is lower than expected.

In summary, if power from the primary power source 650 (“primary power”) is interrupted (e.g., fails), the voltage output from the backup power source 670 (“backup power”) exceeds that of the stepped down primary power and provides the input voltage to the voltage regulator 610 for the supervisor controller 612. In this situation, the supervisor controller 612 switches off the power to the main pump regulators 620 and main pump controllers and other pump circuitry 622, while maintaining power to the clock 630 and the memory 640.

The memory 640 is in communication with the supervisor controller 612 and stores pump settings for the infusion. The memory 640 is coupled to the supervisor controller 612 such that the supervisor can read information from and write information to the memory 640. Moreover, the memory 640 can be used to store the data, instructions, or parameters utilized to support the operation of the infusion pump 100. Memory 640 may be RAM memory, flash memory, EPROM memory, EEPROM memory, or any other form of storage medium known in the art. In the alternative, the memory 640 may be integral to the supervisor controller 612 and/or pump controllers 622. As an example, the supervisor controller 612 and the memory 640 may reside in an application specific integrated circuit (ASIC). Program code for operation of the infusion pump 100 is maintained in the memory 640.

The clock 630 (e.g., a clock or clock circuit) is also in communication with the supervisor controller 612 in order to track the time of the current operating state of an infusion. During interruptions in power from the primary power source 650, the clock 630 continues running using power from the backup power source 670. The clock 630 can physically be a part of the supervisor controller 612 or a separate unit. In one example, the supervisor controller 612 is configured to log a timestamp together with the current status of the infusion (e.g., length of time remaining for the infusion, the dose, rate, etc.). The time at which power from the primary power source 650 is restored can also be retrieved from the clock 630 and stored in the device history. The term “timestamp” may include the time of day and potentially date information and other information typically provided by a clock or clock circuit.

FIG. 6B depicts an example primary power source 650. The illustrated primary power source include three alternative power sources: (1) household batteries 652, e.g., 6 1.5 Volt AA batteries producing a combined 9 Volts; (2) a battery pack 654, e.g., a 4.3 Volt battery pack; and (3) an AC/DC supply 656, e.g., an AD/DC converter that converts mains AC power to 5.5 Volts DC. The battery pack 654 and the AC-DC supply 656 are coupled to a battery charger 658, which is used to charge the backup power 670 via regulator 672.

When power from the AC/DC supply 656 is present, power from the AC/DC supply 656 is used to provide the system voltage 602 and charge the backup power 670 regardless of whether the battery pack 654 or household batteries 652 are installed. When power from the AC/DC supply is not present and the battery pack is installed and providing power, power from the battery pack 654 is used to provide the system voltage 602 and charge the backup power 670. When power from the AC/DC supply is not present and the household batteries 652 are installed and providing power, power from the household batteries 652 is used to provide the system voltage 602 and charge the backup power 670. A power source switch 660 implements the logic for controlling which power source is connected to provide the system voltage 602, e.g., in a similar manner to the circuitry described above for providing primary power 650 and backup power 670 such as diodes 606/608 and a OR'ed junction.

In one example, the infusion pump 100 operation has at least two different states including an initiation state and a current operation state. As used herein, an “initiation state” of operation refers to the state of pump operation at the beginning of an infusion or the restarting/reinitiating of an infusion after power has been restored and a “current operating state” of operation refers to the state of pump operation at a subsequent point in time when the primary power source is interrupted. For example, when power from the primary power source 650 is interrupted, the backup power source 670 powering the clock and the memory stores the time and pump settings for the current operating state of the infusion. Parameters for the current operating state of the infusion refers to infusion parameters, for example, the overall dose the patient has received, the duration of the infusion, the dose rate, etc., at the time of failure of the primary power source 650. In an example, a notification is displayed via user interface 122 indicating that the primary power source has been interrupted. In another example, backup power is not provided to the user interface 122 to conserve energy.

Prior to operation, the operator of the infusion pump 100 of FIGS. 1-5 may be requested to enter parameters to program the infusion pump 100. During the programming of the infusion pump 100 to deliver an infusion to a patient, the operator of the infusion pump 100 may be required to enter many different types of parameters (e.g., patient information, drug information, infusion parameters, etc.) collectively referred to as “pump settings”. For example, when the infusion pump 100 is used to infuse one or more drugs into a patient, the infusion pump 100 may be programmed to specify the drug(s) to be infused, patient data, and infusion data.

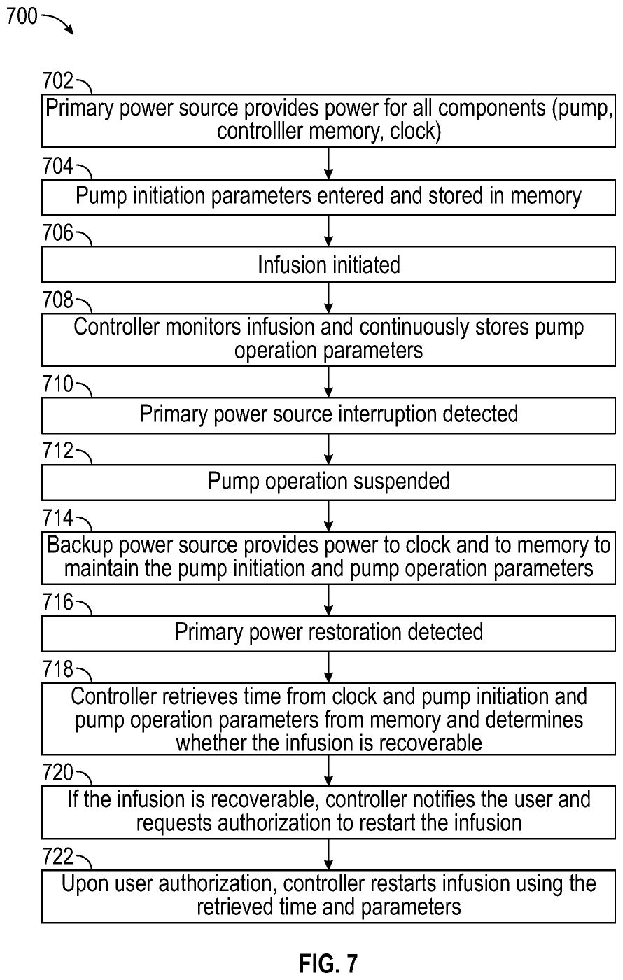

FIG. 7 is a flow chart 700 illustrating example steps of the operation of the infusion pump 100 using the primary power source 650 and backup power source 670 described herein. Modification of the steps for use with other pumps, primary power sources, and backup power sources will be understood by one of skill in the art from the description herein. It will be understood by one of skill in the art that one or more of the steps may be performed concurrently. Additionally, one or more steps may be repeated or omitted.

At step 702, the primary power source 650 provides power to the components of the pump 100. In an example, the primary power source 650 provides power for all components of the pump 100, including the controller 310, memory 640 and the clock 630 along with the pump 106, main pump regulators 620, and other controllers/circuits 622 such as the user interface 122.

At step 704, pump initiation parameters are entered. In an example, a user enters initiation parameters (e.g., patient information and drug delivery parameters) into the pump 100 and the pump 100 stores the entered pump settings. The user may enter the initiation pump settings via the display 124 or buttons 126 of the user interface 122, or by other means (not shown) via a wired or wirelessly coupling to supervisory controller 612. The supervisor controller 612 stores the entered initiation pump settings in memory accessible to the supervisor controller 612 such as memory 640.

At step 706, the supervisor controller 612 initiates the infusion using the initiation pump settings. In an example, the supervisor controller 612 retrieves the initiation pump setting from the memory 640 and uses the retrieved initiation pump settings to initiate the infusion. Power for the infusion operation is provided by the primary power source 650. As described above, the pump 100 includes at least two operation states, e.g., an initiation state and a current operating state. The initiation state is when the infusion is initiated and the current operating state is the current operating state of the infusion in progress, including at the time when the primary power source 650 is interrupted.

At step 708, the supervisor controller 612 continuously monitors pump operation parameters during the infusion and stores the monitored operation parameters in memory. Pump operation parameters include pump setting (e.g., delivery rate) and a duration of the infusion. In an example, the controller 612 monitors the clock 630 and stores the time in memory 640 as an operation parameter. By continuously storing the pump operation parameters and time in the memory 640, the supervisor controller 612 maintains a record of the current operation parameters and time at the moment before pump operation is suspended due to an interruption in power from the primary power source 650.

At step 710, the supervisor controller 612 detects an interruption in power from the primary power source 650. The supervisor controller 612 monitors the voltage output levels of the primary power source 650 and the backup power source 670. In an example, the supervisor controller 612 monitors the primary power source 650 by monitoring both the system voltage 602 (via voltage divider 618) and 5 volt regulated system voltage from regulator 604 (via voltage divider 616). If one of these voltage is within an expected range, the supervisory controller 712 determines that there is no interruption in power and controls the pump 100 for an infusion delivery, including supplying power from the system voltage 602 to the main pump regulators 620 and main pump controllers and other circuitry 622. On the other hand, if neither of these voltages is within the expected range, the supervisor controller 612 detects an interruption in power.

At step 712, the supervisor controller 612 suspends the infusion in response to detecting an interruption in power from the primary power source 650. In an example, the supervisor controller 612 suspends pump operation by turning off the main pump regulators 620 and the main pump controllers and other circuitry 622.

At step 714, the backup power source 670 provides power to the supervisor controller 612, the clock 630, and the memory 640 while power from the primary power source 650 is interrupted. During the power interruption, the supervisor controller 612 performs basic operations including one or more of monitoring power from the backup power source 670 (via voltage divider 614) and monitoring the system voltage 602 (via voltage divider 618) and 5 volt regulated system voltage from regulator 604 (via voltage divider 616) to determine if power from the primary power source has been restored; the clock keeps track of time for use in determining how much time has passed without power from the primary power source 650 upon restoration of power; and the memory 640 maintains the current operating parameters (i.e., the operating parameters of the pump at the time power from the main power source 650 was interrupted and the infusion was suspended).

At step 716, the supervisor controller 612 detects restoration of power from the primary power source 650. The supervisor controller 612 monitors the voltage output levels of the primary power source 650 and the backup power source 670. In an example, the supervisor controller 612 monitors the primary power source 650 by monitoring both the system voltage 602 (via voltage divider 618) and 5 volt regulated system voltage from regulator 604 (via voltage divider 616). If one of these voltages are within an expected range, the supervisory controller 712 determines that power from the primary power source 650 has been restored.

At step 718, the supervisor controller 612 retrieves the time from the clock and pump settings (e.g., initial and current) from the memory. In one example, the supervisor controller 612 determines whether an infusion is recoverable. An infusion may be deemed recoverable (“recoverable infusion”), for example, based on one or more of the following conditions: (1) the motor has not moved since the pump shut off, (2) the cassette has not been removed or reinstalled, or (3) if little time has elapsed since the primary power interruption (e.g., less than three minutes).

At step 720, if the infusion is recoverable, the supervisor controller 612 notifies the user and requests authorization to restart the infusion. In an example, the supervisor controller 612 restores power to the user interface 122 and displays a message requesting user input. The user may respond to the message via the user interface or buttons.

At step 722, the supervisor controller 612 uses the retrieved settings from memory to restart the pump 100 if the infusion is recoverable. If the user authorizes resuming the infusion, the infusion pump will continue the infusion using the current operating parameters stored just prior to interruption of power from the primary power source 650. In an example, the pump settings and infusion parameters do not need to be reentered by the user. In another example, using the retrieved settings from memory to restart the pump bypasses the initiation state of the infusion. In another example, the initiation state of the infusion is bypassed automatically without the need for user approval.

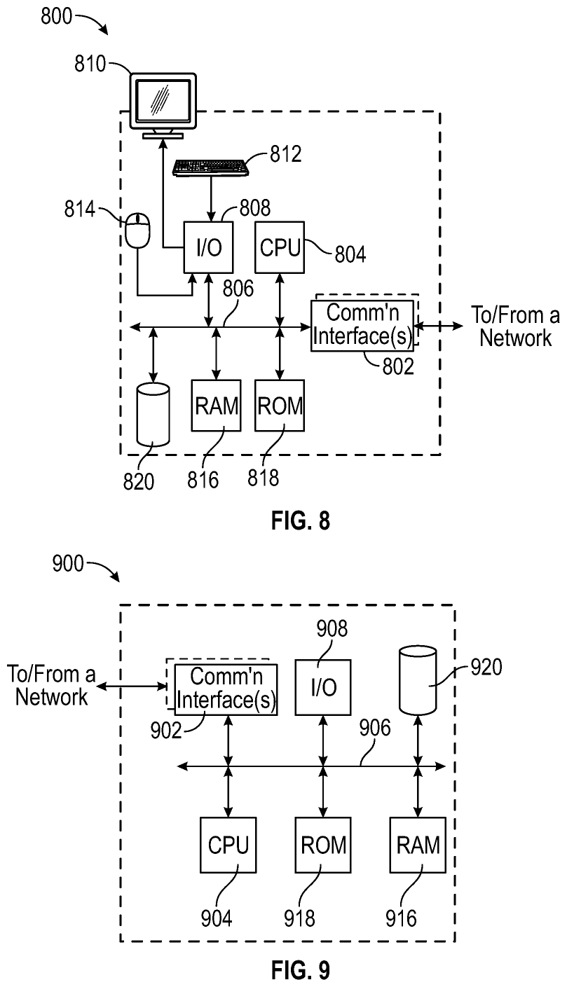

FIGS. 8 and 9 are functional block diagrams illustrating general-purpose computer hardware platforms configured to implement the functional examples described with respect to FIGS. 1-7 as discussed above.

Specifically, FIG. 8 illustrates an example computer platform 800 and FIG. 9 depicts an example computer 900 with user interface elements, as may be used to implement in a personal computer, infusion pump 100, or other type of workstation or terminal device. It is believed that those skilled in the art are familiar with the structure, programming, and general operation of such computer equipment and as a result the drawings should be self-explanatory.

Hardware of an example computer platform 800 (FIG. 8) includes a data communication interface 802 for packet data communication. The computer platform 800 also includes a central processing unit (CPU) 804, in the form of circuitry forming one or more processors, for executing program instructions. The hardware of computer platform 800 typically includes an internal communication bus 806, program and/or data storage 816, 818, and 820 for various programs and data files to be processed and/or communicated by the computer platform 800, although the computer platform 800 often receives programming and data via network communications. In one example, as shown in FIG. 8, the computer platform 800 further includes a video display unit 810 (e.g., a liquid crystal display (LCD) or a cathode ray tube (CRT)), an alphanumeric input device 812 (e.g., a keyboard), a cursor control device 814 (e.g., a mouse), each of which communicate with the internal communication bus 806 via an input/output device (I/O) 808. The hardware elements, operating systems, and programming languages of such computer platforms 800 are conventional in nature, and it is presumed that those skilled in the art are adequately familiar therewith. The computer platform may function as a server that may be implemented in a distributed fashion on a number of similar hardware platforms, to distribute the processing load.

As illustrated in FIG. 9, hardware of a computer type user terminal device 900, such as a PC or tablet computer, similarly includes a data communication interface 902, CPU 904, main memory 916 and 918, one or more mass storage devices 920 for storing user data and the various executable programs, an internal communication bus 906, and an input/output device (I/O) 908.

Aspects of the methods for pump control, as outlined above, may be embodied in programming in general purpose computer hardware platforms (such as described above with respect to FIGS. 8 and 9), e.g., in the form of software, firmware, or microcode executable by a networked computer system such as a server or gateway, and/or a programmable nodal device. Program aspects of the technology may be thought of as “products” or “articles of manufacture” typically in the form of executable code and/or associated data that is carried on or embodied in a type of machine readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software, from one computer or processor/controller into another, for example, from a processor/controller central processing unit (CPU) 804 of the system 800 and/or from a pump controller 310 of a peristaltic infusion pump 100 to a computer or software of another system (not shown). Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to one or more of “non-transitory,” “tangible” or “storage” media, terms such as computer or machine “readable medium” refer to any medium that participates in providing instructions to a processor for execution.

Hence, a machine-readable medium may take many forms, including but not limited to, a tangible storage medium, a carrier wave medium, or physical transmission medium. Non-transitory storage media include, for example, optical or magnetic disks, such as any of the storage devices in any computer(s) or the like. It may also include storage media such as dynamic memory, for example, the main memory of a machine or computer platform. Tangible transmission media include coaxial cables, copper wire, and fiber optics, including the wires that include a bus within a computer system. Carrier-wave transmission media can take the form of electric or electromagnetic signals, or acoustic or light waves such as those generated during radio frequency (RF) and light-based data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

Program instructions may include a software or firmware implementation encoded in any desired language. Programming instructions, when embodied in machine readable medium accessible to a processor of a computer system or device, render the computer system or device into a special-purpose machine that is customized to perform the operations specified in the program performed by the controller 310 of the pump 100.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is ordinary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 105 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that includes a list of elements does not include only those elements but may include other elements not expressly listed or inherent to such process, method, article, or apparatus. An element preceded by “a” or “an” does not, without further constraints, preclude the existence of additional identical elements in the process, method, article, or apparatus that includes the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing describes what is considered to be the best mode and other examples, it is understood that various modifications may be made and that the subject matter disclosed herein may be implemented in various forms and examples, and that they may be applied in numerous applications, only some of which have been described herein. It is intended by the following claims to claim any and all modifications and variations that fall within the true scope of the present concepts.

What is claimed is:

1. An infusion pump comprising:
a pump configured to deliver an infusion;
a clock configured to track time;
a memory;
a controller in operative communication with the pump, the clock, and the memory that controls operation of the infusion pump, retrieves time from the clock, and stores pump settings in the memory, wherein the pump settings include a duration of the infusion;
a primary power source configured to provide power to the pump, the clock, the memory, and the controller during the infusion; and
a backup power source configured to provide power to the clock and the memory during an interruption in power from the primary power source to maintain the pump settings.

2. The infusion pump of claim 1, wherein upon restoration of power from the primary power source, the controller is configured to retrieve a current time from the clock and the pump settings from the memory.

3. The infusion pump of claim 2, wherein, upon authorization by a user, the controller restarts the pump and continues the infusion using the retrieved pump settings.

4. The infusion pump of claim 3, wherein the pump settings do not need to be reentered to restart the pump.

5. The infusion pump of claim 3, wherein the infusion pump's operation comprises at least an initiation state and a current operating state and wherein the initiation state of the infusion is bypassed when the pump is restarted.

6. The infusion pump of claim 5, wherein the initiation state is bypassed automatically.

7. The infusion pump of claim 3, wherein the infusion pump's operation comprises at least an initiation state and a current operating state, the initiation state comprises initial parameters, and the initial parameters include patient information, drug information, infusion information, or a combination thereof.

8. The infusion pump of claim 1, further comprising:
a voltage regulator coupled to the primary power source, the backup power source, and the controller.

9. The infusion pump of claim 8, wherein the voltage regulator is configured to stabilize an input voltage to the controller.

10. The infusion pump of claim 1, wherein the backup power source is a rechargeable battery.

11. A method of operating an infusion pump, the method comprising:
providing an infusion using the infusion pump;
tracking time with a clock;
storing pump settings for the infusion in a memory, the pump settings including a duration of the infusion;
detecting interruption in power from a primary power source for the infusion pump;
suspending the infusion upon detecting the interruption in power from the primary power source; and
powering the clock and the memory with a backup power source while the infusion is suspended to maintain the pump settings for the infusion.

12. The method of claim 11, further comprising:
retrieving, upon restoration of the power from the primary power source, the pump settings from the memory.

13. The method of claim 12, further comprising:
resuming the infusion, upon user authorization, using the retrieved pump settings such that the infusion continues from an operating state of the pump at the time of detecting the interruption in power from the primary power source.

14. The method of claim 13, wherein the pump settings do not need to be reentered.

15. The method of claim 13, wherein an initiation state of the infusion is bypassed.

16. The method of claim 15, wherein the initiation state is bypassed automatically.

17. The method of claim 15, wherein the initiation state comprises initial parameters, wherein the initial parameters include patient information, drug information, infusion information, or a combination thereof.

18. The method of claim 11, further comprising:
stabilizing an input voltage from the primary power source to a controller of the infusion pump.

19. A non-transitory computer-readable storage medium storing instructions for controlling operation of an infusion pump, wherein the instructions, when executed by a controller, cause the infusion pump to perform operations comprising:
providing an infusion using the infusion pump;
tracking time with a clock;
storing pump settings for the infusion in a memory, the pump settings including a duration of the infusion;
detecting interruption in power from a primary power source for the infusion pump;
suspending the infusion upon detecting the interruption in power from the primary power source; and
powering the clock and the memory with a backup power source while the infusion is suspended to maintain the pump settings for the infusion.

20. The non-transitory computer-readable storage medium of claim 19, wherein the instructions cause the infusion pump to perform the further operations of:
retrieving, upon restoration of the power from the primary power source, the pump settings from the memory; and
resuming the infusion, upon user authorization, using the retrieved pump settings such that the infusion continues from an operating state of the pump at the time of detecting the interruption in power from the primary power source.
